(19) United States
(12) Patent Application Publication
(10) Pub. No.: US 2017/0143590 A1
(43) Pub. Date: May 25, 2017

(54) Medical Pacifier

(71) Applicant: Byron Smith, Moscow, TN (US)

(72) Inventor: Byron Smith, Moscow, TN (US)

(21) Appl. No.: 14/947,269

(22) Filed: Nov. 20, 2015

(51) Int. Cl. A61J 17/00; A61M 15/08; A61M 16/20; A61M 16/10; A61M 16/12

(52) U.S. Cl. CPC A61J 17/006; A61M 16/1005; A61M 16/104; A61M 16/12; A61M 16/201; A61M 15/08; A61M 2202/0208; A61M 2202/0283; A61M 2210/1035

(57) ABSTRACT

A medical pacifier for supplying gases such as oxygen, nitrous oxide or albuterol to the user, with the novel feature of a control valve included in the pacifier to allow the operator to direct the flow of the gas to the oral cavity, the nasal area, or both the oral cavity and nasal area of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### DESCRIPTION

### Field of the Invention

This disclosure relates a medical pacifier that is capable of supplying entrained gas such as oxygen to an infant wherein said gas can be directed to the infant's oral cavity, nasal area or both oral cavity and nasal area by means of a flow adjustment valve within the medical pacifier.

### BACKGROUND OF THE INVENTION

Medical pacifiers have been developed to aid in the supply of entrained gases or medicines to an infant. These pacifiers generally involve a hollow nipple with a plurality of holes in the nipple that can be inserted into an infant's mouth, wherein the hollow nipple contains the gas or medicine. In the case of an entrained gas, the nipple is connected to a gas supply tube through which the gas flows in order for the gas to enter the infant's oral cavity. In the case of a medicine, the hollow nipple is capable of being filled with a medicine and while the infant sucks on the nipple, the medicine is drawn through the holes in the nipple, which then enters the infant's oral cavity.

In a second general design of medical pacifiers for the supply of gases to an infant, the pacifier is attached to a gas supply tube and the gas is directed from the tube, through passageways in the pacifier, and the gas is expelled from the passageways in the pacifier in the nasal area of the infant.

The present disclosure reveals a medical pacifier with a hollow nipple with a plurality of holes in the nipple and a plurality of passageways through the body of the pacifier, along with a control valve in the medical pacifier such that the control valve directs the flow of the gas through the pacifier to the nipple, to the passageways or through both the nipple and the passageways, thus allowing the operator to control the direction of flow of the gas as desired.

### SUMMARY OF THE INVENTION

The present disclosure reveals a medical pacifier that is adjustable such that a gas supply tube can be attached to the medical pacifier in order to supply gases such as oxygen, nitrous oxide or albuterol to the user with the additional novel feature that the pacifier also comprises a control valve that allows the user to select where to direct the flow of the gas; to the oral cavity through the nipple of the pacifier, through passageways up to the nasal area of the user, or to both the oral cavity and nasal area of the user.

The nipple can have multiple embodiments. In the first embodiment the gas passes directly through the nipple and through a single hole at the end of the nipple. In the second embodiment, the gas passes into the nipple and is then diffused through a plurality of holes in the nipple.

### BRIEF DESCRIPTION OF THE SEVERAL VIEWS OF THE DRAWING

FIG. 1 is a view of the parts of the medical pacifier including the gas connection, front portion, body, guard, and both embodiments of the nipple;

FIG. 2A is a profile view of the internal portion of the body of the medical pacifier including the plurality of passageways upward, the passage to the nipple, the guard, and the interior portion of the body;

FIG. 2B is a view of body in association with the control valve of the front portion;

FIG. 2C is a portrayal of the control valve;

FIG. 2D is a view of a type of adjustment means;

FIG. 3 is a view of the medical pacifier including the gas connection adjustment means, guard, and plurality of passageways upward;

FIG. 4 is a view of the first embodiment of the nipple of the medical pacifier including the gas jet protruding through the tip;

FIG. 5 is a view of the second embodiment of the nipple of the medical pacifier including a plurality of holes in the nipple that allow the gas to be dispersed from the medical pacifier;

FIG. 6 is a profile cut-away of the medical pacifier including the first embodiment of the nipple, including the gas jet; and

FIG. 7 is a profile cut-away of the medical pacifier including the second embodiment of the nipple.

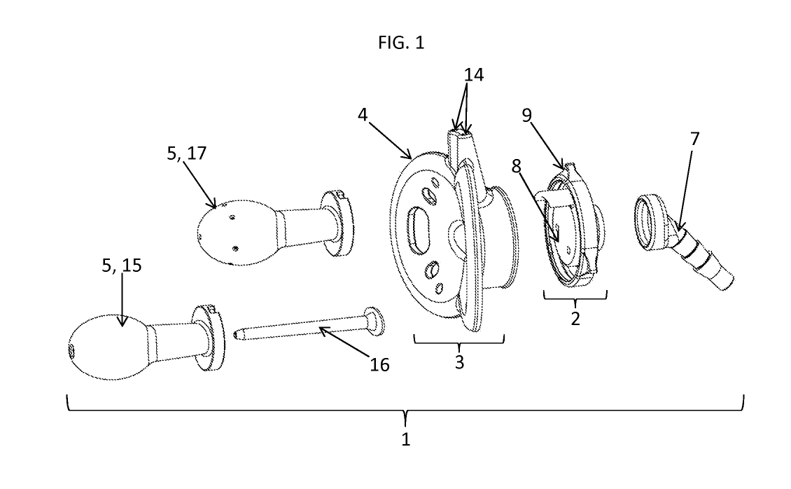

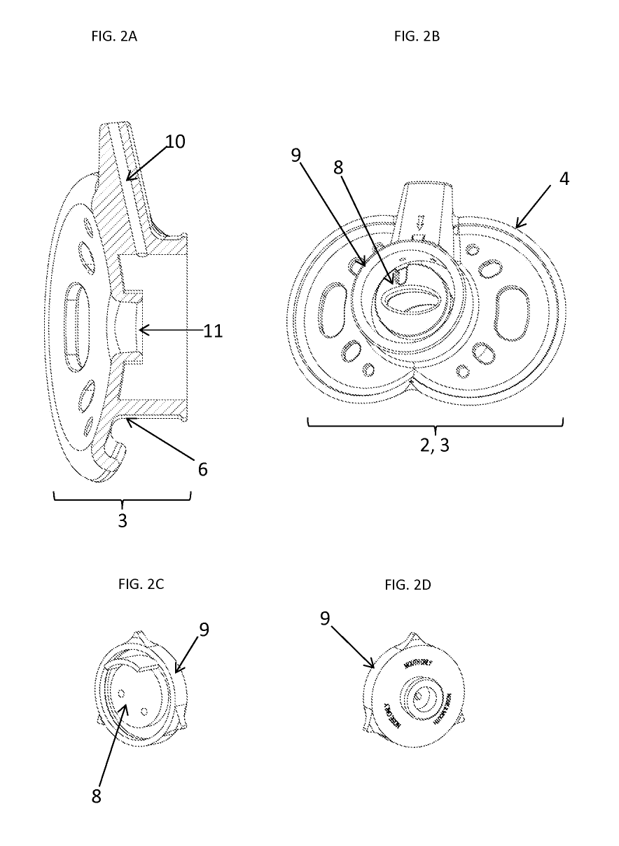

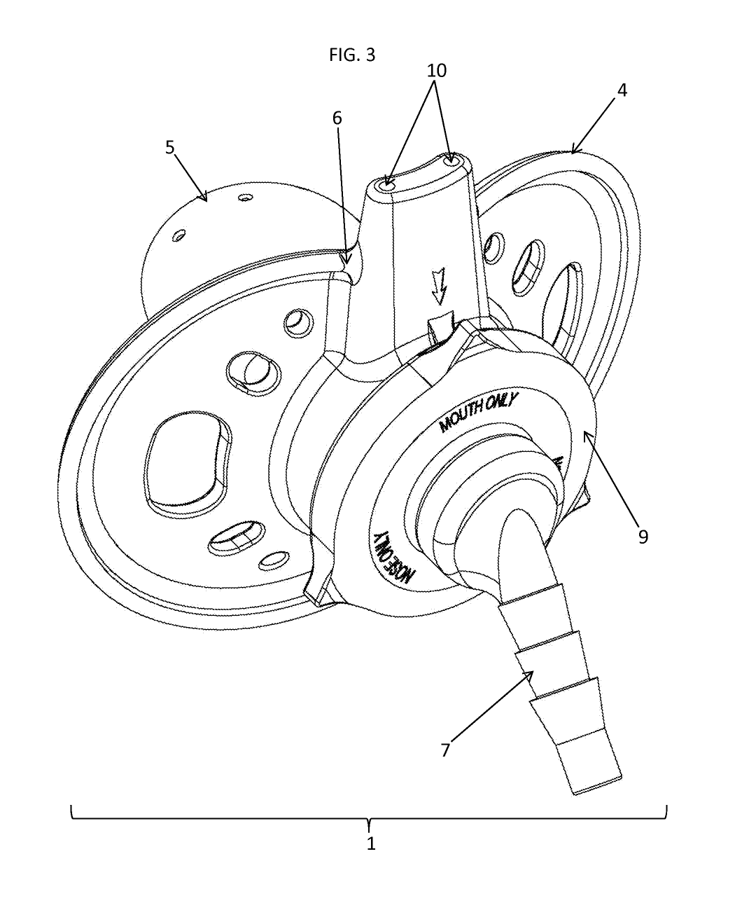

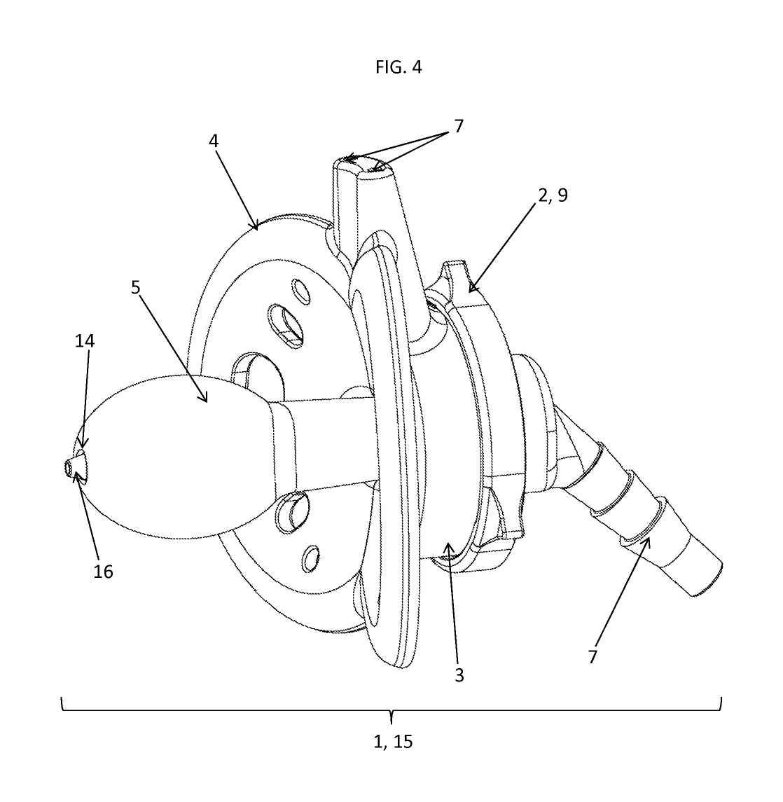

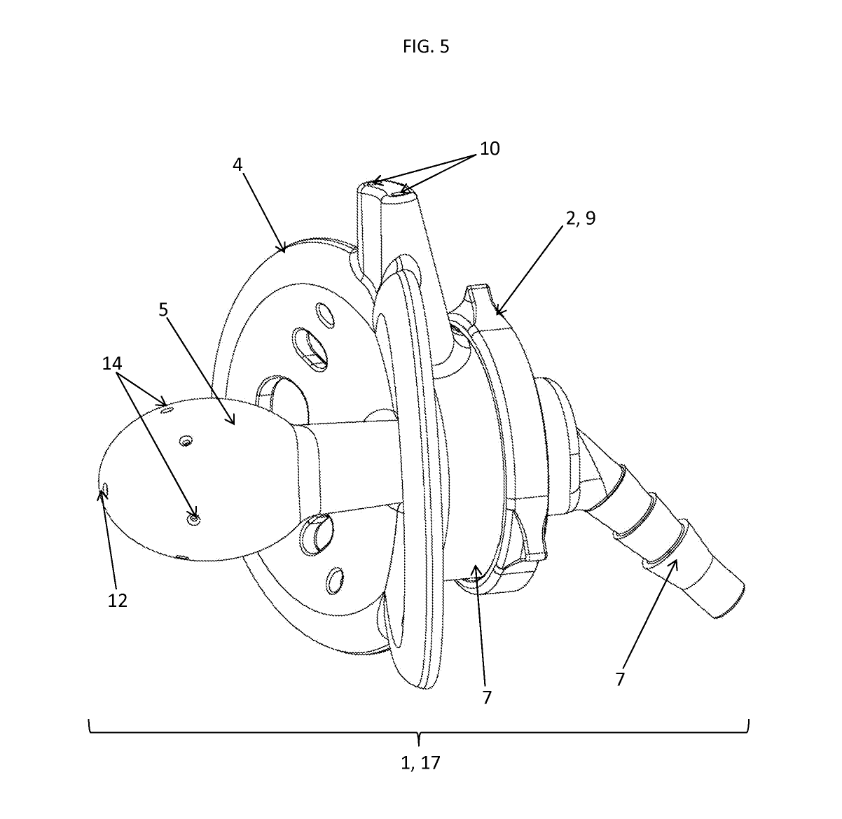

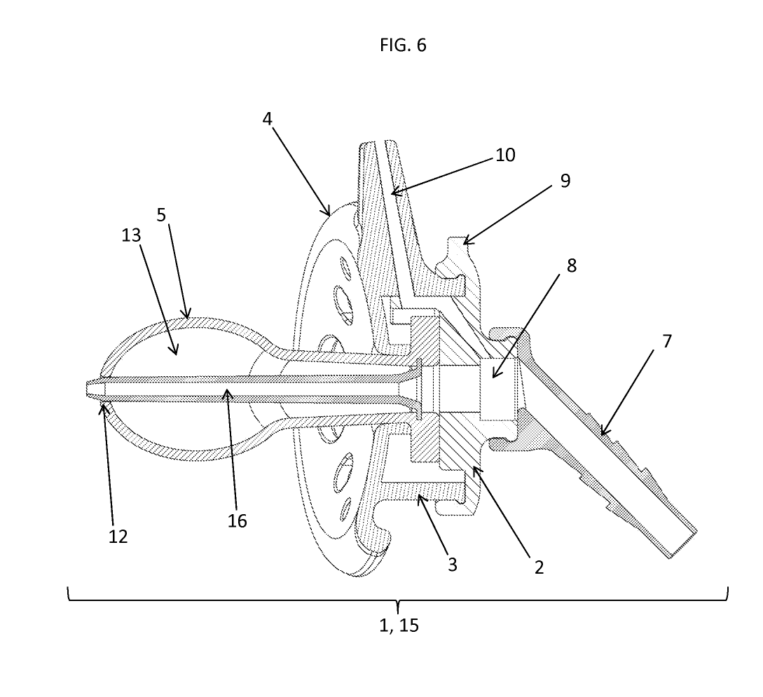

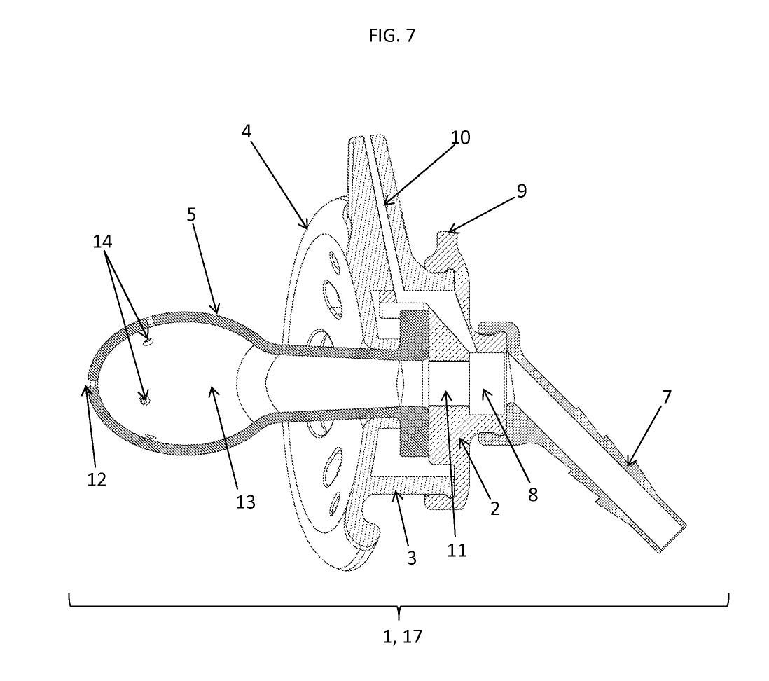

### DETAILED DESCRIPTION OF THE INVENTION

The present disclosure reveals a medical pacifier 1 for the supply of gases such as oxygen, nitrous oxide or albuterol to the user's oral cavity, nasal cavity, or oral and nasal cavity, comprising a front portion 2, a body 3, a guard 4, and a nipple 5. The front portion 2 is attached to the body 3, with the guard 4 attached around the exterior 6 of the body 3, and the nipple 5 being attached to and extending from the body 3 in the direction opposite the front portion 2, such that the nipple 5 is capable of being inserted into a user's mouth.

The front portion 2 comprises a gas connection 7, a control valve 8 and an adjustment means 9 for the control valve 8 wherein such adjustment means 9 is comprised of a knob, dial, or switch. The body 3 comprises a plurality of passageways extending upward 10, a passage to the nipple 11, and the guard 5 around the exterior 6 of the body 3.

The nipple 5 comprises a tip 12 and a hollow portion 13 such that gas can pass from the gas connection 7 through the front portion 2, be directed by the control valve 8 through the passage to the nipple 11, into the nipple 5, and collect in the hollow portion 13. The nipple 5 further comprises a plurality of holes 14 to allow the gas to pass from the hollow portion 13 into the user's mouth.

With the manipulation of the adjustment means 9 the control valve 8 is capable of directing gas to the plurality of passageways that extend upward 10, the passage to the nipple 11, or to both the plurality of passageways that extend upward 10 and the passage to the nipple 11.

The nipple 5 has several embodiments. In the first embodiment 15, the plurality of holes 14 in the nipple 5 is comprised of a single hole at the tip 12 of the nipple 5. In the first embodiment 15, a gas jet 16 may also be inserted into the hollow portion 13 of the nipple 5 to focus the flow of the gas through the nipple 5.

In the second embodiment 17 of the nipple 5, the plurality of holes 14 in the nipple 5 allow the gas to flow through the nipple 5 and exit through the plurality of holes 14 at a diffuse pressure.

What is claimed:

1. A medical pacifier for the supply of gases such as oxygen, nitrous oxide or albuterol to the user's oral cavity, or nasal cavity oral and nasal cavity comprising:
a front portion, a body, a guard, and a nipple;
the front portion being attached to the body, with the guard attached to the exterior of the body, and the nipple being attached to and extending from the body in the direction opposite the front portion, such that the nipple is capable of being inserted into a user's mouth;
the front portion comprising a gas connection, a control valve and an adjustment means for the control valve;
the body comprising a plurality of passageways extending upward, a passage to the nipple, and the guard around the exterior of the body;
the nipple comprising a tip and a hollow portion such that gas can pass through the front portion, be directed through the passage to the nipple, in to the nipple, and collect in the hollow portion of the nipple;
the nipple further comprising a plurality of holes to allow the gas to pass from the hollow portion into the user's mouth; and
the control valve being capable of directing gas to the plurality of passageways that extend upward, the passage to the nipple, or to both the plurality of passageways that extend upward and the passage to the nipple.

2. The medical pacifier of claim 1 wherein the plurality of holes in the nipple comprise a single hole at a tip of the nipple.

3. The medical pacifier of claim 2 further comprising a gas jet in the hollow portion of the nipple that focuses the flow of the gas through the nipple.

4. The medical pacifier of claim 1 wherein the plurality of holes in the nipple allow the gas to flow through the nipple and exit the pacifier at a diffuse pressure.
